(19) United States
(12) Patent Application Publication
(10) Pub. No.: US 2003/0212412 A1
(43) Pub. Date: Nov. 13, 2003

(54) Intra-bronchial obstructing device that permits mucus transport

(75) Inventors: David H. Dillard, Redmond, WA (US); Clifton A. Alferness, Redmond, WA (US); Lauri J. DeVore, Seattle, WA (US); Hugo X. Gonzalez, Woodinville, WA (US)

Correspondence Address: KNOBBE MARTENS OLSON & BEAR LLP, 2040 MAIN STREET, FOURTEENTH FLOOR, IRVINE, CA, 92614, US

(73) Assignee: Spiration, Inc.

(21) Appl. No.: 10/143,353

(22) Filed: May 9, 2002

(51) Int. Cl. A61F 11/00

(52) U.S. Cl. 606/108

(57) ABSTRACT

An obstructive device prevents air from being inhaled into a lung portion to collapse the lung portion while providing mucus transport from the lung portion. When placed in an air passageway serving the lung portion, the obstructing member defines a pathway for mucus transport between the obstructing member and the air passageway. The device may include a tubular-shaped anchor to retain the device in the air passageway. A pathway for mucus transport is provided between a portion of the anchor and a portion of the obstructing device.

### BACKGROUND OF THE INVENTION

The present invention is generally directed to a device, system, and method for treating Chronic Obstructive Pulmonary Disease (COPD). The present invention is more particularly directed to a providing an intra-bronchial obstruction while permitting mucus transport and clearance from a collapsed lung portion.

COPD has become a major cause of morbidity and mortality in the United States over the last three decades. COPD is characterized by the presence of airflow obstruction due to chronic bronchitis or emphysema. The airflow obstruction in COPD is due largely to structural abnormalities in the smaller airways. Important causes are inflammation, fibrosis, goblet cell metaplasia, and smooth muscle hypertrophy in terminal bronchioles.

The incidence, prevalence, and health-related costs of COPD are on the rise. Mortality due to COPD is also on the rise. In 1991, COPD was the fourth leading cause of death in the United States and had increased 33% since 1979.

COPD affects the patient's whole life, producing increasing disability. It has three main symptoms: cough; breathlessness; and wheeze. At first, breathlessness may be noticed when running for a bus, digging in the garden, or walking uphill. Later, it may be noticed when simply walking in the kitchen. Over time, it may occur with less and less effort until it is present all of the time.

COPD is a progressive disease and currently has no cure. Current treatments for COPD include the prevention of further respiratory damage, pharmacotherapy, and surgery. Each is discussed below.

The prevention of further respiratory damage entails the adoption of a healthy lifestyle. Smoking cessation is believed to be the single most important therapeutic intervention. However, regular exercise and weight control are also important. Patients whose symptoms restrict their daily activities or who otherwise have an impaired quality of life may require a pulmonary rehabilitation program including ventilatory muscle training and breathing retraining. Long-term oxygen therapy may also become necessary.

Pharmacotherapy may include bronchodilator therapy to open up the airways as much as possible or inhaled beta-agonists. For those patients who respond poorly to the foregoing or who have persistent symptoms, ipratropium bromide may be indicated. Further, courses of steroids, such as corticosteroids, may be required. Lastly, antibiotics may be required to prevent infections and influenza and pneumococcal vaccines may be routinely administered. Unfortunately, there is no evidence that early, regular use of pharmacotherapy will alter the progression of COPD.

About 40 years ago, it was first postulated that the tethering force that tends to keep the intrathoracic airways open was lost in emphysema and that by surgically removing the most affected parts of the lungs, the force could be partially restored. Although the surgery was deemed promising, the procedure was abandoned.

The lung volume reduction surgery (LVRS) was later revived. In the early 1990's, hundreds of patients underwent the procedure. However, the number of procedures has declined because Medicare stopped reimbursing for LVRS. The procedure is currently under review in controlled clinical trials. However, preliminary data indicates that patients benefited from the procedure in terms of an increase in forced expiratory volume, a decrease in total lung capacity, and a significant improvement in lung function, dyspnea, and quality of life.

Improvements in pulmonary function after LVRS have been attributed to at least four possible mechanisms. These include enhanced elastic lung recoil, correction of ventilation/perfusion mismatch, improved efficiency of respiratory muscaulature, and improved right ventricular filling.

Lastly, lung transplantation is also a therapeutic option. Today, COPD is the most common diagnosis for which lung transplantation is considered. Unfortunately, this consideration is given for only those with advanced COPD. Given the limited availability of donor organs, lung transplant is far from being available to all patients.

The inventions disclosed and claimed in U.S. Pat. Nos. 6,258,100 and 6,293,951, both of which are incorporated herein by reference, provide an improved therapy for treating COPD. The therapy includes non-surgical apparatus and procedures for reducing lung volume by permanently obstructing the air passageway that communicates with the portion of the lung to be collapsed. An obstruction is placed in the air passageway that prevents inhaled air from flowing into the portion of the lung to be collapsed. Lung volume reduction with concomitant improved pulmonary function may be obtained without the need for surgery. Various other apparatus and techniques may exist for permanently obstructing the air passageway.

Mucus transport in normal airways includes mucus transport by the mucociliary mechanism and coughing mechanism. It carries bacteria out of the lungs and prevents pneumonia. Although various apparatus and methods have been conceived for permanently obstructing an air passageway and collapsing a portion of a lung, none addresses a potential complication where the permanent obstruction may interfere with mucus transport by mucociliary or coughing transport mechanism.

In view of the foregoing, there is a need in the art for a new and improved apparatus and method for permanently obstructing an air passageway that minimizes the potential complication to or interference with mucus transport. The present invention is directed to a device, system, and method which provide such an improved apparatus and method for treating COPD.

### SUMMARY OF THE INVENTION

The present invention provides an apparatus and method for use in a treatment regime that treats COPD by reducing the size of a lung by permanently collapsing at least a portion of the lung. The invention permits mucus transport past an intra-bronchial obstructing device used to collapse the lung portion.

The present invention provides an intra-bronchial device adapted to be placed in an air passageway to collapse a lung portion associated with the air passageway. The device includes an obstructing member that both prevents air from being inhaled into the lung portion to collapse the lung portion, and permits mucus transport from the lung portion. Further, the obstructing member, when placed in the air passageway, may define at least one peripheral pathway providing for mucus transport. At least one peripheral pathway providing for mucus transport may be between a portion of the exterior perimeter surface of the obstructing member and a portion of the interior surface of the air passageway. The obstructing member may allow air to pass from the lung portion to be collapsed. The obstructing member may include a flexible membrane impervious to air flow.

In accordance with an additional embodiment of the invention, the invention provides an intra-bronchial device adapted to be placed in an air passageway to collapse a lung portion associated with the air passageway. The device of the additional embodiment comprises an anchor that retains the device in the air passageway, and an obstructing member carried by the anchor that prevents air from being inhaled into the lung portion to collapse the lung portion and being arranged to permit mucus transport from the lung portion. The anchor may be arranged to maintain continuous contact with the interior perimeter of the air passageway. The anchor may comprise a ring-shaped member having an interior surface. Furthermore, the anchor may comprise a generally tubular member. The obstructing member may be mounted on the anchor to define at least one peripheral pathway that provides for mucus transport. In an alternative embodiment, the obstructing member is mounted on the anchor to form at least one peripheral pathway between a portion of an interior perimeter surface of the anchor and a portion of the exterior perimeter surface of the obstructing member. In a further alternative embodiment, the anchor provides for re-epithelialization, allowing mucus transport along at least one pathway between the anchor and the obstructing member. The obstructing member may allow air to pass from the lung portion to be collapsed. The obstructing member may also include a flexible membrane impervious to air flow, the membrane being secured at selected areas around the interior perimeter of the anchor to form at least one mucus transport pathway.

The present invention still further provides a method for reducing the size of a lung by collapsing a portion of the lung while permitting mucus transport. The method includes the step of placing an obstructing member in an air passageway communicating with the portion of the lung to be collapsed, the obstructing member being arranged to permit mucus transport past the obstructing member while precluding air from being inhaled into the portion of the lung. The placing step may include providing at least one peripheral pathway between an interior perimeter surface portion of the air passageway and an exterior perimeter surface portion the obstructing member. The obstructing member of the method may allow air to pass from the lung portion to be collapsed. The obstructing member of the method may further include a flexible membrane impervious to air flow.

In yet another embodiment, the invention provides a method for reducing the size of a lung by collapsing a portion of the lung while permitting mucus transport. The method includes the steps of placing an anchor in an air passageway communicating with the portion of the lung, and mounting an obstructing member on the anchor to define at least one pathway that permits mucus transport past the obstructing member. The obstructing member precludes air from being inhaled into the portion of the lung. The mounting step may include providing at least one peripheral pathway between an interior perimeter surface portion of the anchor and an exterior perimeter surface portion for permitting mucus transport. The obstructing member of the method may allow air to pass from the lung portion to be collapsed. The obstructing member of the method may further includes a flexible membrane impervious to air flow. The anchor of the method may comprise a ring-shaped member having an interior surface. Furthermore, the anchor may comprise a generally tubular member.

In a further embodiment, the invention provides an apparatus for reducing the size of a lung by collapsing a portion of the lung while permitting mucus transport. The apparatus includes an obstructing means for obstructing an air passageway communicating with the portion of the lung, the obstructing means being dimensioned for insertion into the air passageway, for precluding air to be inhaled through the air passageway into the lung portion, and for permitting mucus transport from the lung portion while maintaining the preclusion of inhaled air from flowing into the lung portion to collapse the portion of the lung. The obstructing means may be dimensioned to define at least one peripheral pathway for providing mucus transport when placed in the air passageway. The apparatus may further include an anchor means for anchoring the obstructing member in the air passageway. The obstructing means may be mounted on the anchor means to define at least one peripheral pathway between the anchoring means and the obstructing means for permitting mucus transport.

In yet a further embodiment, the invention provides a system for reducing the size of a lung by collapsing a portion of the lung while permitting mucus transport. The system comprises an intra-bronchial device adapted to be placed in an air passageway to collapse a lung portion associated with the air passageway, the device including an obstructing member that prevents air from being inhaled into the lung portion while permitting mucus transport from the lung portion, and an apparatus that places the intra-bronchial device in the air passageway.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a simplified sectional view of a thorax illustrating a healthy respiratory system;

FIG. 2 is a simplified sectional view of a thorax illustrating the mucus transport system in a respiratory system;

FIG. 3 is a sectional view similar to FIG. 1 but illustrating a respiratory system suffering from COPD, and an initial step in placing an obstructing member;

FIG. 4 illustrates a further step in a method for placement of an obstructing member in a bronchial sub-branch;

FIG. 5 is a perspective view, partly in section, and to an enlarged scale, illustrating an obstructing member positioned in an air passageway for sealing the lung portion;

FIG. 6 illustrates additional details concerning a bronchial wall, a mucus layer, and an obstructing member;

FIG. 7 is a longitudinal section view that illustrates additional detail related to the contact areas formed by the obstructing member and mucus layer;

FIG. 8 illustrates additional details of an obstructing member;

FIG. 9 is a cross-sectional view illustrating an obstructing member placed in an air passageway and permitting mucus transport;

FIG. 10 illustrates a stent-like anchor and an obstructing member in position within an air passageway;

FIG. 11 illustrates a stent-like anchor disposed on a bronchial wall, with the obstructing member not being shown to better illustrate the re-epithelialization process;

FIG. 12 illustrates a cross-sectional view of an air passageway with a stent-like anchor and an obstructing member placed in an air passageway, and providing for mucus transport;

FIG. 13 illustrates a longitudinal sectional view of a stent-like anchor and an obstructing member placed in an air passageway, taken through two coupling areas; and

FIG. 14 illustrates a longitudinal sectional view of a stent-like anchor and an obstructing member placed in an air passageway, taken midway through two relatively flat areas of the obstructing member.

### DETAILED DESCRIPTION OF THE PREFERRED EMBODIMENTS

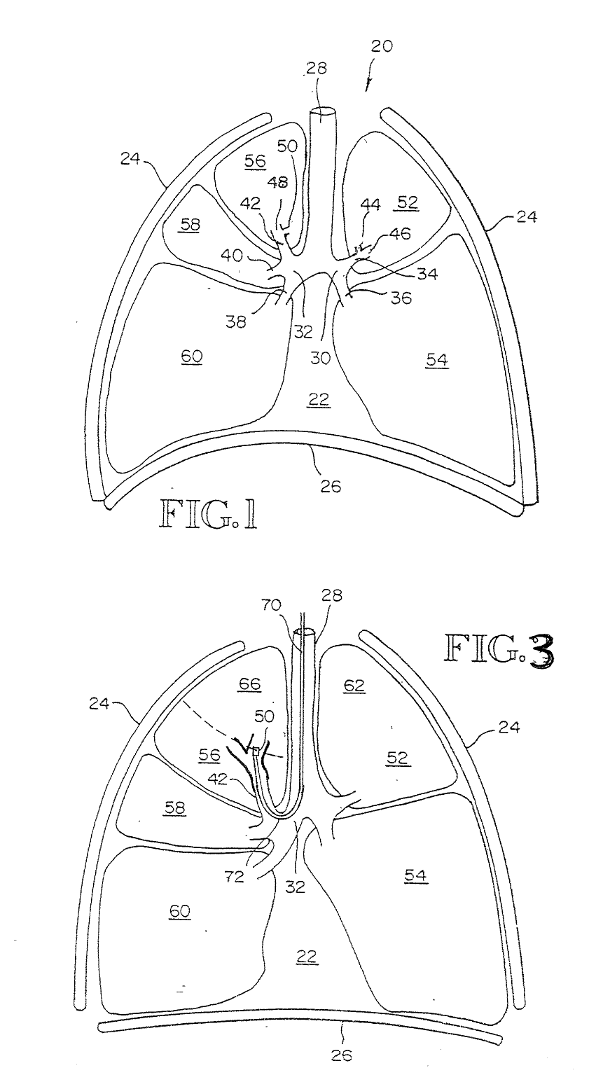

Referring now to FIG. 1, it is a sectional view of a healthy respiratory system. The respiratory system 20 resides 25 within the thorax 22, which occupies a space defined by the chest wall 24 and the diaphragm 26.

The respiratory system 20 includes the trachea 28, the left mainstem bronchus 30, the right mainstem bronchus 32, the bronchial branches 34, 36, 38, 40, and 42 and sub-branches 44, 46, 48, and 50. The respiratory system 20 further includes left lung lobes 52 and 54 and right lung lobes 56, 58, and 60. Each bronchial branch and sub-branch communicates with a respective different portion of a lung lobe, either the entire lung lobe or a portion thereof. As used herein, the term “air passageway” is meant to denote either a bronchi or bronchiole, and typically means a bronchial branch or sub-branch which communicates with a corresponding individual lung lobe or lung lobe portion to provide inhaled air thereto or conduct exhaled air therefrom.

Characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes to fill with air. When the individual exhales, the diaphragm returns to its original arched condition to decrease the volume of the thorax. The decreased volume of the thorax causes a positive pressure within the thorax which in turn causes exhalation of the lung lobes.

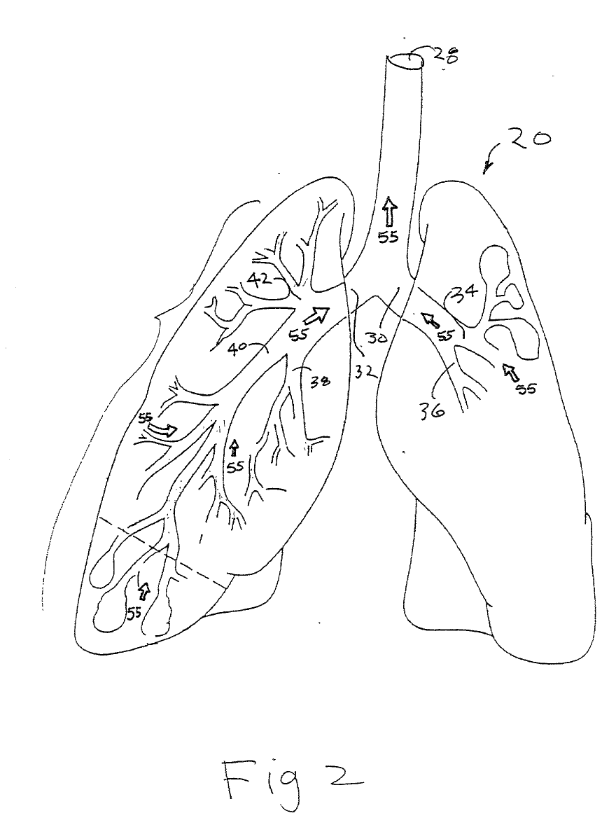

FIG. 2 illustrates the mucus transport system in a normal lung. Many pollution particles are inhaled as a person breathes, and the air passageways function as a very effective filter. The mucus transport system 55 functions as a self-cleaning mechanism for all air passageways, including the lungs. The mucus transport system 55 is a primary method for mucus clearance from distal portions of the lungs, and further constitutes a primary immune barrier for the lungs. The surface of air passageways is formed with respiratory epithelium (or epithelial membrane), which is covered with cilia and coated with mucus. As part of the mucus transport system 55, the mucus entraps many inhaled particles and moves them toward the larynx 28. Mucus transport system 55 includes the metachronal ciliary beat of cilia on the respiratory epithelium that moves a continuous carpet of mucus and entrapped particles from the distal portions of the lungs past the larynx 28 and to the pharynx for expulsion from the respiratory system. The mucus transport system 55 also includes the coughing transport mechanism. The explosive expiration of a cough helps clear the lungs of secretions and foreign bodies. “Mucus transport” as used in the specifications, including the description and claims, includes the mucociliary transport system and the coughing transport mechanism.

In contrast to the healthy respiratory system of FIG. 1, FIG. 3 illustrates a respiratory system suffering from COPD. Here it may be seen that the lung lobes 52, 54, 56, 58, and 60 are enlarged and that the diaphragm 26 is not arched but substantially straight. Hence, this individual is incapable of breathing normally by moving the diaphragm 26. Instead, in order to create the negative pressure in the thorax 22 required for breathing, this individual must move the chest wall outwardly to increase the volume of the thorax. This results in inefficient breathing causing these individuals to breathe rapidly with shallow breaths.

It has been found that the apex portions 62 and 66 of the upper lung lobes 52 and 56, respectively, are most affected by COPD. Hence, bronchial sub-branch obstructing devices are generally employed for treating the apex 66 of the right, upper lung lobe 56. However, as will be appreciated by those skilled in the art, the present invention may be applied to any lung portion without departing from the present invention. As will be further appreciated by those skilled the in art, the present invention may be used with any type of obstructing member to permit mucus transport. The inventions disclosed and claimed in U.S. Pat. Nos. 6,258,100 and 6,293,951, both of which are incorporated herein by reference, provide an improved therapy for treating COPD by obstructing an air passageway using an intrabronchial valve or plug. The present invention may be used with the apparatus, system, and methods of these patents as will be briefly described in conjunction with the disclosure of the preferred embodiments of the present invention.

The insertion of an obstructing member treats COPD by deriving the benefits of lung volume reduction surgery without the need of performing the surgery. The treatment contemplates permanent collapse of a lung portion. This leaves extra volume within the thorax for the diaphragm to assume its arched state for acting upon the remaining healthier lung tissue. As previously mentioned, this should result in improved pulmonary function due to enhanced elastic recoil, correction of ventilation/perfusion mismatch, improved efficiency of respiratory musculature, and improved right ventricle filling. The present invention supports the use of intra-bronchial plugs to treat COPD by allowing mucus transport to continue after insertion of the obstructing device, thus reducing entrapment of bacteria distal to the obstructing device.

FIG. 3 also illustrates a step in COPD treatment using an obstructing-member. Treatment is initiated by feeding a conduit or catheter 70 down the trachea 28, into the right mainstem bronchus 32, into the bronchial branch 42 and into and terminating within the sub-branch 50. The sub-branch 50 is the air passageway that communicates with the lung portion 66 to be treated. The catheter 70 is preferably formed of flexible material such as polyethylene. Also, the catheter 70 is preferably preformed with a bend 72 to assist the feeding of the catheter from the right mainstem bronchus 32 into the bronchial branch 42, or could be deformed to conform to different curvatures and angles of the bronchial tree.

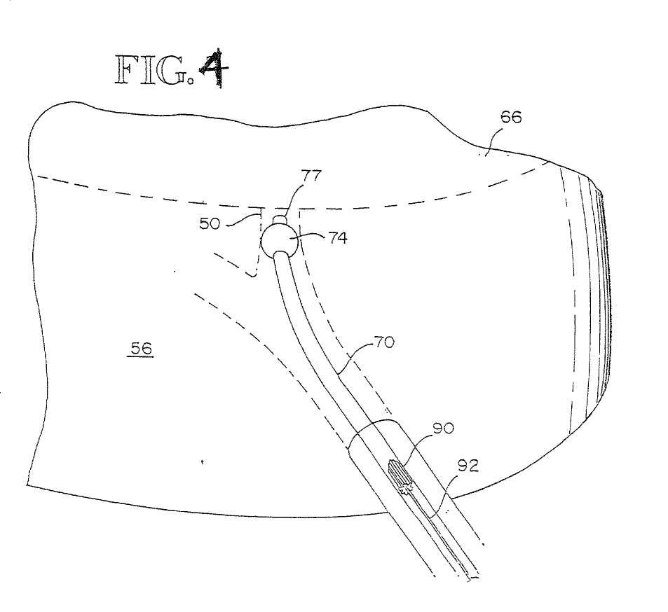

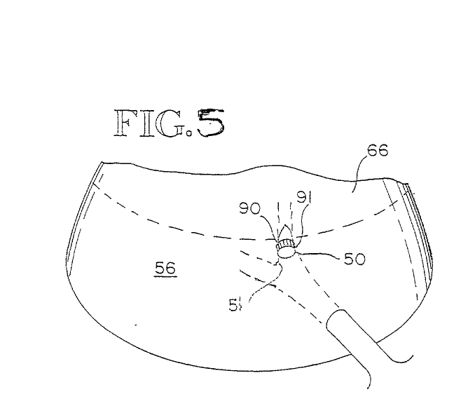

FIG. 4 illustrates a further step in a method for placing an obstructing member 90 in a bronchial sub-branch using a catheter. The invention disclosed herein is not limited to use with the particular method illustrated herein. Catheter 70 may be used alone to perform the insertion, may be extended from a bronchoscope, or used in conjunction with a bronchoscope. For purposes of this description, the insertion will be described with reference to only the catheter 70. The invention disclosed herein is not limited to use with the particular method illustrated herein. Catheter 70 includes an optional inflatable sealing member 74 for use with a vacuum to collapse lung portion 66 prior to insertion of obstructing member 90. The obstructing member 90 may be formed of resilient or collapsible material to enable the obstructing member 90 to be fed through the conduit 70 in a collapsed state. The stylet 92 is used to push the obstructing member 90 to the end 77 of the catheter 70 for placing the obstructing member 90 within the air passageway 50 adjacent to the lung portion 66 to be permanently collapsed. Optional sealing member 74 is withdrawn after obstructing member 90 is inserted.

FIG. 5 illustrates the obstructing device in place within air passageway 50. Obstructing member 90 has expanded upon placement in the air passageway 50 to seal the air passageway 50. This causes the lung portion 66 to be maintained in a permanently collapsed state. The obstructing member 90 may be any shape suitable for accomplishing its purpose, and may be a solid material or a membrane.

More specifically, the obstructing member 90 has an outer dimension 91, and when expanded, enables contact with the air passageway inner dimension 51. This seals the air passageway upon placement of the obstructing member 90 in the air passageway 50 for maintaining the lung portion 66 in the collapsed state. As described below, obstructing member 90 is arranged to permit mucus transport from collapsed lung 66 while sealing the air passageway 50.

Alternatively, the lung portion 66 may be collapsed using vacuum prior to placement of obstructing member 90, or it may be collapsed by sealing the air passageway 50 with obstructing member 90. Over time, the air within the lung portion 66 will be absorbed by the body and result in the collapse of lung portion 66. Alternatively, obstructing member 90 may include a one-way valve allowing air to escape from lung portion 66 but precluding air from being inhaled. Lung portion 66 will then collapse, and the valve will prevent air from being inhaled.

A function of the intra-bronchial device disclosed and claimed in this specification, including the description and the claims, is described in terms of collapsing a lung portion associated with an air passageway to reduce lung volume. In some lungs, a portion of a lung may receive air from collateral air passageways. Obstructing one of the collateral air passageways may reduce the volume of the lung portion associated with the air passageway, but not completely collapse the lung portion as that term may be generally understood. In other situations, obstruction of an air passageway may not result in a complete collapse of the lung portion, but still may provide the benefits of lung volume reduction. As used in the description and claims herein, the meaning of “collapse” includes a complete collapse of a lung portion, a partial collapse of a lung portion, and a reduction of lung volume.

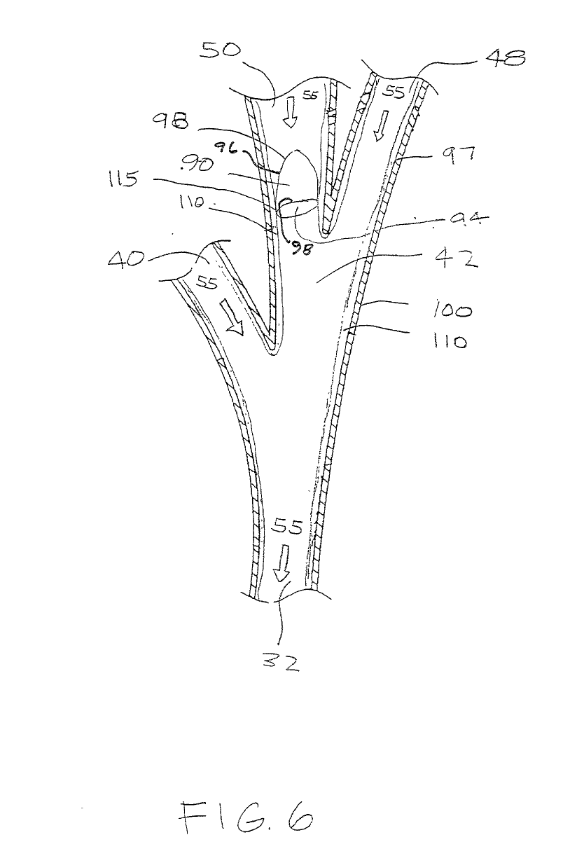

FIG. 6 illustrates additional details about a bronchial wall, a mucus layer, and an obstructing member. Bronchial wall 100 includes an epithelial membrane 97 with cilia (not shown), also known as respiratory epithelium or epithelial layer, on the inside or air passageway side. The epithelial membrane is coated with mucus layer 110, which traps inhaled particles. The inhaled particles are moved out of the respiratory system by the mucus transport system 55 as described in FIG. 2.

In this embodiment, obstructing member 90 generally has conical configuration, and may be hollow. More specifically, the obstructing member 90 includes a segmented periphery that renders it generally circular at its base, referred to herein as circular base cross-section 94. The obstructing member 90 further includes a circumferential, generally conical sidewall 96 that extends from the outer periphery of generally circular cross-section base 94. The sidewall 96 has an exterior perimeter surface 98 that defines the outer periphery of the obstructing member 90. The obstructing member 90 is arranged so that a portion of its outer periphery contacts mucus layer 110 of bronchial wall 100 at a plurality of contact areas 115 to form a loose seal that precludes air from moving past obstructing member 90, while permitting mucus transport system 55 to continue.

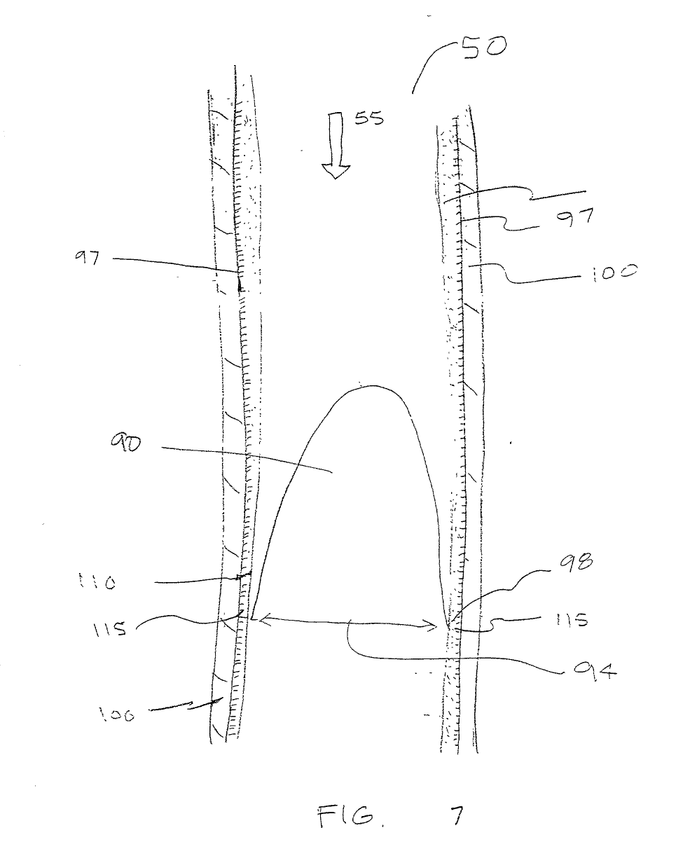

FIG. 7 is a longitudinal section view that illustrates additional detail related to the contact areas 115 formed by the intersection of obstructing member 90 and mucus layer 110.

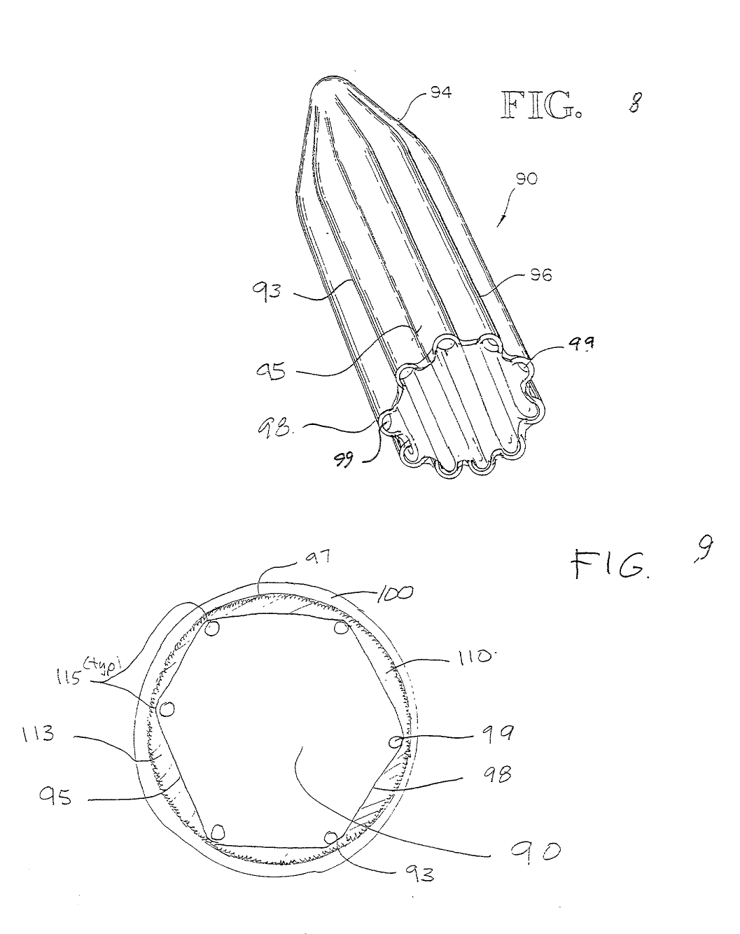

FIG. 8 illustrates additional details of a preferred embodiment of an obstructing member. The obstructing member 90 includes a plurality of inner resilient reinforcement ribs 99. The quantity, composition, and location of inner resilient reinforcement ribs 99 may be varied as necessary, taking into consideration the size of the air passageway to be sealed, the materials comprising the obstructing member 90, and other relevant factors. Exterior perimeter surface 98 may comprise a membrane.

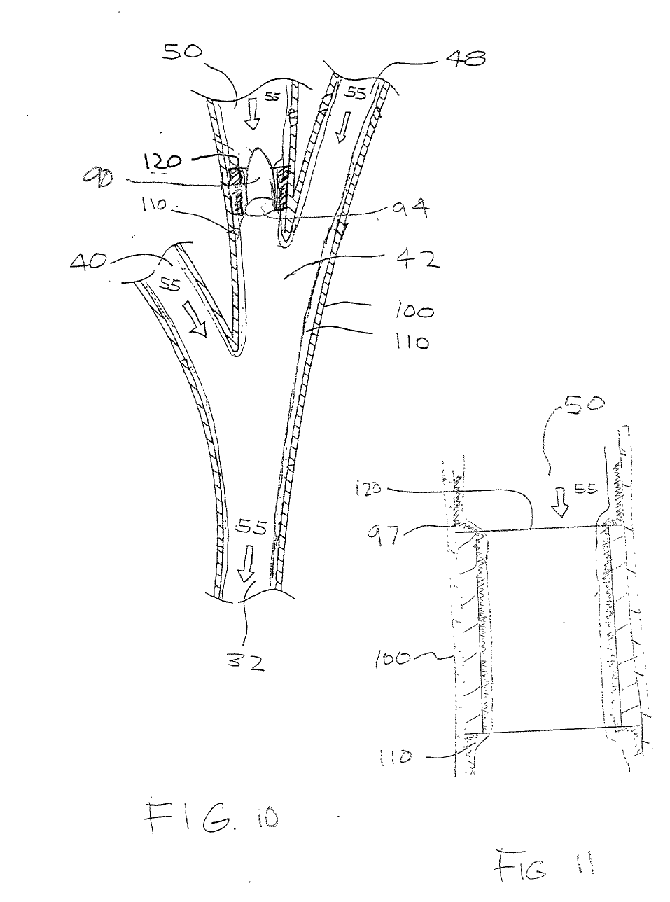

FIG. 9 is a cross-sectional view of the obstructing member 90 of FIG. 8 placed in an air passageway and providing for mucus transport. When the obstructing member 90 is placed in an air passageway, the reinforcement ribs 99 expand to create a series of relatively flat areas 95 and ridges 93 around the exterior perimeter surface 98. The ridges 93 press loosely against the epithelial membrane 97 and bronchial wall 100 to form contact areas 115. The ridges 93 hold the obstructing member 90 in position within the bronchial sub-branch by contact areas 115 on the epithelial membrane 97 and the underlying bronchial wall 100. The relatively flat areas 95 of exterior perimeter surface 98 and the relatively curved wall of bronchial wall 100 form peripheral pathways 113 for mucus 110 to flow past the obstructing member 90, thus permitting mucus transport 55 from the lung portion to be collapsed.

FIGS. 10-13 illustrate an alternative embodiment where the intra-bronchial device includes an obstructing member carried on a stent-like anchor having a ring shape. FIG. 10 illustrates the stent-like anchor 120 and the obstructing member 90 positioned within air passageway 50. The stent-like anchor 120 and obstructing member 90 may each be made of any compatible materials and in any configuration known in the art suitable for placement in an air passageway by any suitable technique known in the art. Stent-like anchor 120 is anchored on bronchial wall 100 by a forced fit. To that end, the stent-like anchor 120 may be balloon expandable as is known in the art, or may be self-expanding. In a preferred embodiment, stent-like anchor 120 and obstructing member 90 are coupled at a plurality of coupling areas 130 before placement into air passageway 50. They may be coupled by any means appropriate for the materials used, method of installation selected, patient requirements, and degree of permanency selected. Coupling methods may include friction, adhesive and mechanical joint. In an alternative embodiment, stent-like anchor 120 and obstructive member 90 may be coupled during placement in air passageway 50.

In a further alternative embodiment, stent-like anchor 120 may be comprise a serpentined, small tubular member. A majority of the length of the small tubular member is orientated longitudinally, and bends are formed were the small tubular member reverses longitudinal direction. The longitudinal portions of the serpentined small tubular member are arranged to contact the interior perimeter of the air passageway upon deployment of the anchor. The bends are arranged to be displaced centrally of the interior perimeter of the air passageway upon deployment of the anchor, and are further arranged to provide a mucus pathway between the peripheral portion of the bend and the interior perimeter of the air passageway.

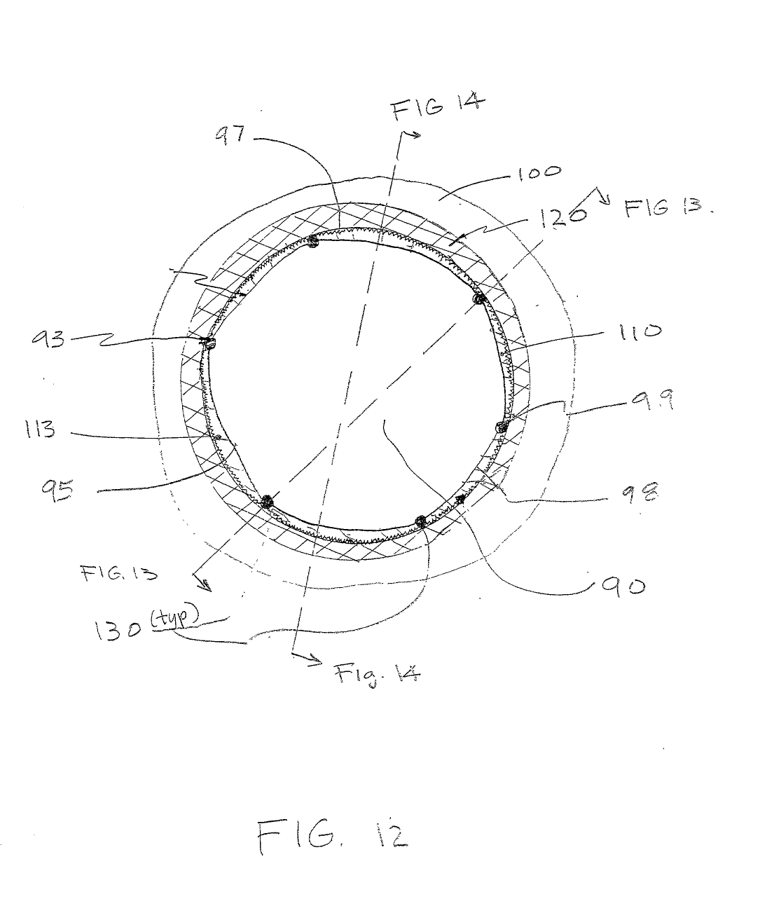

FIG. 11 illustrates the stent-like anchor 120 disposed on bronchial wall 100, with obstructing member 50 not shown for clarity. Initially, the physical characteristics of stent-like anchor 120 may block the epithelial membrane 97 and mucus transport system 55. FIG. 11 illustrates the body's normal process of re-epithelialization. Epithelial tissue 110 and cilia will grow on stent-like anchor 120 over time, and permit mucus transport.

In an alternative embodiment, stent-like anchor 120 may be first placed in the air passageway and disposed on the bronchial wall 100 without obstructing member 50 being coupled to it. The epithelial layer is allowed to become established across the stent-like anchor 120 over time. Then the obstructing member 50 is coupled to the stent-like anchor 120.

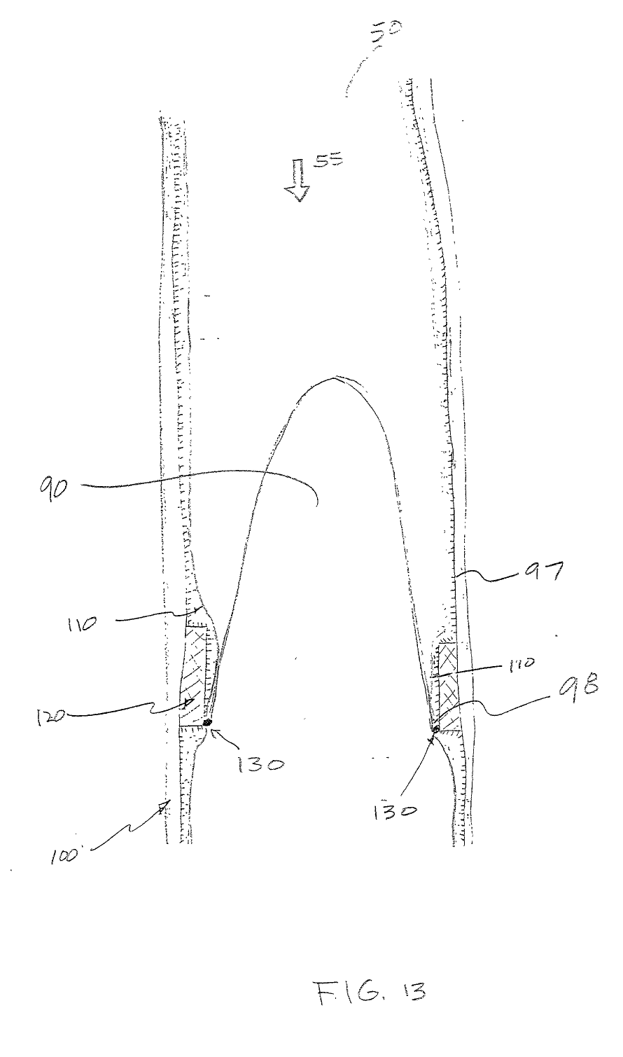

FIG. 12 is a transverse cross-section view of the stent-like anchor of FIG. 11 and the obstructing member of FIG. 10 in place and providing for mucus transport. FIG. 12 is similar to FIG. 9, with the addition of the stent-like anchor 120 and a plurality of coupling areas 130 for this alternative embodiment. Re-epithelialization is illustrated across stent-like anchor 120. Coupling areas 130 couple obstructing member 90 to stent-like anchor 120 at a plurality of locations. In a manner similar to the embodiment depicted in FIG. 9, the exterior perimeter surface 98 of obstructing member 90 has a shape that includes a series of relatively flat areas 95 between coupling areas 130. A relatively flat area 95 of outer periphery 91 and a portion of the relatively curved wall of stent-like anchor 120 form a peripheral pathway 113 for mucus 110 to flow past obstructing member 90, thus permitting mucus transport 55 from the lung portion to be collapsed.

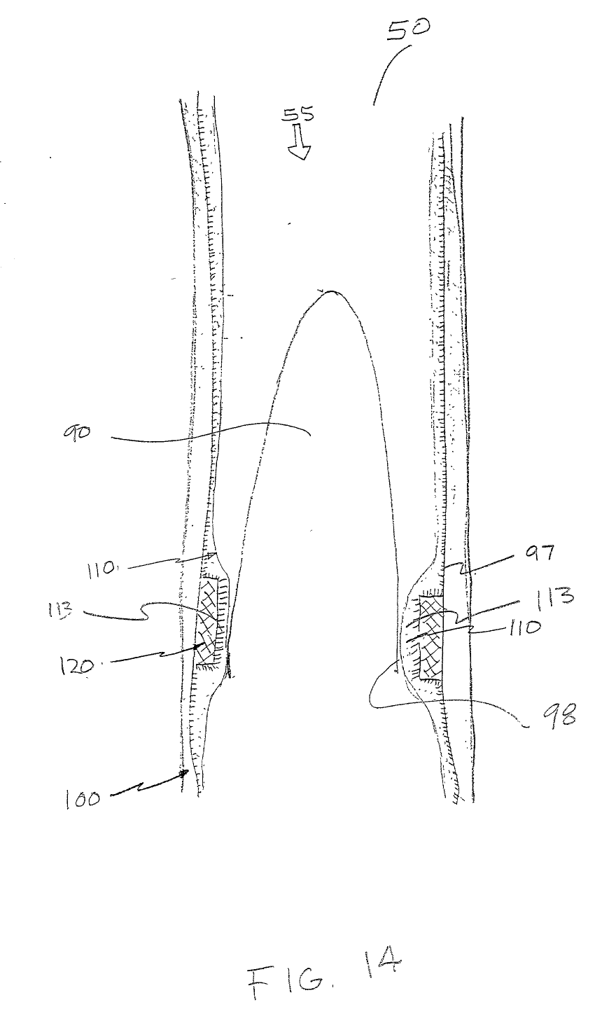

FIG. 13 is a longitudinal sectional view of a stent-like anchor and an obstructing member placed in an air passageway, taken through two coupling areas. Coupling areas 130 may reduce re-epithelialization and physically obstruct mucus transport system 55. An alternative embodiment may use the minimum number of coupling areas 130 necessary to carry obstructive member 90.

FIG. 14 is a longitudinal sectional view of a stent-like anchor and an obstructing member placed in an air passageway, taken midway through two relatively flat areas of obstructing member. FIG. 14 illustrates peripheral pathways 113 formed between a relatively flat area 95 and a relatively curved wall portion of stent-like anchor 120 for re-epithelialization and for mucus layer 110. These peripheral pathways 113 permit mucus transport 55 from the lung portion to be collapsed past obstructing member 90.

As can thus be seen from the foregoing, the present invention provides an intra-bronchial device, system, and method for permitting mucus transport from a lung being treated for COPD by lung volume reduction. Mucus transportation is achieved by providing an obstructive member that prevents air from being inhaled into the lung portion being treated while providing a pathway suitable for mucus transport.

While particular embodiments of the present invention have been shown and described, modifications may be made, and it is therefore intended in the appended claims to cover all such changes and modifications which fall within the true spirit and scope of the invention.

What is claimed is:

1. An intra-bronchial device adapted to be placed in an air passageway to collapse a lung portion associated with the air passageway, the device comprising an obstructing member that prevents air from being inhaled into the lung portion to collapse the lung portion and that permits mucus transport from the lung portion.

2. The device of claim 1, wherein the obstructing member, when placed in the air passageway, defines at least one peripheral pathway providing for mucus transport.

3. The device of claim 1, wherein the obstructing member, when placed in the air passageway, defines at least one peripheral pathway providing for mucus transport between a portion of the exterior perimeter surface of the obstructing member and a portion of the interior surface of the air passageway.

4. The device of claim 1, wherein the obstructing member allows air to pass from the lung portion to be collapsed.

5. The device of claim 1, wherein the obstructing member includes a flexible membrane impervious to air flow.

6. An intra-bronchial device adapted to b e placed in an air passageway to collapse a lung portion associated with the air passageway, the device comprising:
an anchor that retains the device in the air passageway; and
an obstructing member carried by the anchor that prevents air from being inhaled into the lung portion to collapse the lung portion and being arranged to permit mucus transport from the lung portion.

7. The device of claim 6, wherein the anchor is arranged to maintain continuous contact with the interior perimeter of the air passageway.

8. The device of claim 6, wherein the anchor comprises a ring-shaped member having an interior surface.

9. The device of claim 6, wherein the anchor comprises a generally tubular member.

10. The device of claim 6, wherein the obstructing member is mounted on the anchor to define at least one peripheral pathway that provides for mucus transport.

11. The device of claim 6, wherein the obstructing member is mounted on the anchor to form at least one peripheral pathway between an interior perimeter surface portion of the anchor and an exterior perimeter surface portion of the obstructing member.

12. The device of claim 6, wherein the obstructing member allows air to pass from t he lung portion to be collapsed.

13. The device of claim 6, wherein the anchor provides for re-epithelialization, allowing mucus transport along at least one pathway between the anchor and the obstructing member.

14. The device of claim 6, wherein the obstructing member includes a flexible membrane impervious to air flow, the membrane being secured at selected areas around the interior perimeter of the anchor to form at least one mucus transport pathway.

15. A method of reducing the size of a lung by collapsing a portion of the lung while permitting mucus transport, the method including the step of placing an obstructing member in an air passageway communicating with the portion of the lung to be collapsed to permit mucus transport past the obstructing member while precluding air from being inhaled into the portion of the lung.

16. The method of claim 15, wherein the placing step includes providing at least one peripheral pathway between an interior perimeter surface portion of the air passageway and an exterior perimeter surface portion the obstructing member.

17. The method of claim 15, wherein the obstructing member allows air to pass from the lung portion to be collapsed.

18. The method of claim 15, wherein the obstructing member further includes a flexible membrane impervious to air flow.

19. A method of reducing the size of a lung by collapsing a portion of the lung while permitting mucus transport, the method including the steps of:
placing an anchor in an air passageway communicating with the portion of the lung; and
mounting an obstructing member on the anchor to define at least one pathway that permits mucus transport past the obstructing member, the obstructing member precludes air from being inhaled into the portion of the lung.

20. The method of claim 19, wherein the mounting step includes providing at least one peripheral pathway between an interior perimeter surface portion of the anchor and an exterior perimeter surface portion of the obstructing member for permitting mucus transport.

21. The method of claim 19, wherein the obstructing member allows air to pass from the lung portion to be collapsed.

22. The method of claim 19, wherein the obstructing member includes a flexible membrane impervious to air flow.

23. The method of claim 19, wherein the anchor comprises a ring-shaped member having an interior surface.

24. The method of claim 19, wherein the anchor comprises a generally tubular member.

25. An apparatus for reducing the size of a lung by collapsing a portion of the lung while permitting mucus transport, the apparatus comprising:
obstructing means for obstructing an air passageway communicating with the portion of the lung, the obstructing means being dimensioned for insertion into the air passageway, for precluding air to be inhaled through the air passageway into the lung portion, and for permitting mucus transport from the lung portion while maintaining the preclusion of inhaled air from flowing into the lung portion to collapse the portion of the lung.

26. The apparatus of claim 25, wherein the obstructing means is dimensioned to define at least one peripheral pathway for providing mucus transport when placed in the air passageway.

27. The apparatus of claim 25, further including anchor means for anchoring the obstructing member in the air passageway.

28. The apparatus of claim 27, wherein the obstructing means is mounted on the anchor means to define at least one peripheral pathway between the anchoring means and the obstructing means for permitting mucus transport.

29. A system for reducing the size of a lung by collapsing a portion of the lung while permitting mucus transport, the system comprising:
an intra-bronchial device adapted to be placed in an air passageway to collapse a lung portion associated with the air passageway, the device including an obstructing member that prevents air from being inhaled into the lung portion while permitting mucus transport from the lung portion; and
an apparatus that places the intra-bronchial device in the air passageway.
